(19) United States
(12) Patent Application Publication
(10) Pub. No.: US 2021/0228241 A1
(43) Pub. Date: Jul. 29, 2021

(54) Polyaxial Lateral Offset Connector

(71) Applicants: Christopher Harrod, Baton Rouge, LA (US); Peter Newton, La Jolla, CA (US); Khaled Kebaish, Baltimore, MD (US); Han Jo Kim, New York, NY (US); Keenan O'Brien, Broadlands, VA (US)

(72) Inventors: Christopher Harrod, Baton Rouge, LA (US); Peter Newton, La Jolla, CA (US); Khaled Kebaish, Baltimore, MD (US); Han Jo Kim, New York, NY (US); Keenan O'Brien, Broadlands, VA (US)

(21) Appl. No.: 17/050,990

(22) PCT Filed: May 2, 2019
(86) PCT No.: PCT/US2019/030392

§ 371 Date: Oct. 27, 2020

Related U.S. Application Data
(60) Provisional application No. 62/665,869, filed on May 2, 2018.

(51) Int. Cl. A61B 17/70

(52) U.S. Cl. CPC A61B 17/7005; A61B 17/704; A61B 17/7034

(57) ABSTRACT

A polyaxial offset lateral connector includes a body, an insert, and a connecting shaft. The body has a base that defines a receptacle and walls that extend from the base to define a saddle. The insert includes a cup and a pin that extends from a bottom surface of the cup. The connecting shaft has a connection portion and a neck extending from the connecting shaft to a ball. The ball is received within the receptacle of the body and defines a pin passage therethrough. The pin is the insert is received within the pin passage of the ball to prevent translation of the connecting shaft relative to the body.

### CROSS-REFERENCE TO RELATED APPLICATIONS

The present application claims the benefit of the filing date of U.S. Provisional Application No. 62/665,869, filed May 2, 2018 entitled POLYAXIAL LATERAL OFFSET CONNECTOR, the disclosure of which is hereby incorporated herein by reference.

### BACKGROUND

### 1. Technical Field

The present disclosure relates to spinal constructs and, more specifically, to laterally offset connectors for use with spinal contructs.

### 2. Discussion of Related Art

The spinal column is a complex system of bones and connective tissues that provide support for the human body and protection for the spinal cord and nerves. The adult spine is comprised of an upper portion and a lower portion. The upper portion contains twenty-four discrete bones, which are subdivided into three areas including seven cervical vertebrae, twelve thoracic vertebrae and five lumbar vertebrae. The lower portion is comprised of the sacral and coccygeal bones. The cylindrical shaped bones, called vertebral bodies, progressively increase in size from the upper portion downwards to the lower portion.

An intervertebral disc along with two posterior facet joints cushion and dampen the various translational and rotational forces exerted upon the spinal column. The intervertebral disc is a spacer located between two vertebral bodies. The facets provide stability to the posterior portion of adjacent vertebrae. The spinal cord is housed in the canal of the vertebral bodies. It is protected posteriorly by the lamina. The lamina is a curved surface with three main protrusions. Two transverse processes extend laterally from the lamina, while the spinous process extends caudally and posteriorly. The vertebral bodies and lamina are connected by a bone bridge called the pedicle.

The spine is a flexible structure capable of a large range of motion. There are various disorders, diseases and types of injury, which restrict the range of motion of the spine or interfere with important elements of the nervous system. The problems include, but are not limited to, scoliosis, kyphosis, excessive lordosis, spondylolisthesis, slipped or ruptured discs, degenerative disc disease, vertebral body fracture, and tumors. Persons suffering from any of the above conditions typically experience extreme or debilitating pain and often times diminished nerve function. These conditions and their treatments can be further complicated if the patient is suffering from osteoporosis, or bone tissue thinning and loss of bone density.

Spinal fixation apparatuses are widely employed in surgical processes for correcting spinal injuries and diseases. When the disc has degenerated to the point of requiring removal, there are a variety of interbody implants that are utilized to take the place of the disc. These include interbody spacers, metal cages and cadaver and human bone implants. In order to facilitate stabilizing the spine and keeping the interbody in position, other implants are commonly employed, such as bone screws and rods. Depending on the pathology and treatment, a surgeon will select the appropriate spinal rod material and size, specifically, the cross-sectional diameter.

One growing trend seen in post-surgical treatment of a patient's spine is the incidence of proximal junctional kyphosis (PJK), which is typically an adult spinal deformity surgical outcome if the lumbar lordosis and thoracic kyphosis are not properly restored post-surgery. PJK appears at or above the cranial-most thoracic level treated. Even though PJK most commonly occurs in the thoracic region of the spine, it can also occur in various spinal regions and may occur above or below the instrumented levels and may impact the next adjacent level or two that is not instrumented. This type of failure is called adjacent level failure. Symptoms of PJK and adjacent level failure include pain, neurological deficit, ambulatory difficulty and poor maintenance of sagittal balance. For patients that exhibit these symptoms, often the only treatment is an additional surgery. The incidence rate of PJK may be upward of 50% in long construct, instrumented fusion cases. Factors contributing to this condition are the end vertebrae selection, facet violation, weakened structural support due to significant soft tissue disruption, extensive junctional paraspinal musculature dissection and loss of integrity of the posterior tension band.

One thought to address the problem of PJK, which is caused by the accelerated degeneration of the joint capsules and smaller articular processes at one or two levels above or below the junctional region, is to decrease the structural rigidity of the construct at the top of the construct just below the proximal junction, thereby providing a transition from the relatively stiff instrumented spine to the more flexible non-instrumented spine.

Spinal rods are typically made of cobalt chrome, stainless steel, or titanium alloy. However in order to transition to a less stiff construct at the top, other less rigid materials may be employed to provide the desired stiffness. A continuing need exists for an improved device, an improved system, and an improved method for performing spine surgery that does not create additional morbidity post-surgical treatment.

### SUMMARY

In an aspect of the present disclosure, a polyaxial offset lateral connector includes a body, an insert, and a connecting shaft. The body has a base that defines a receptacle and walls that extend from the base to define a saddle. The insert includes a cup and a pin that extends from a bottom surface of the cup. The connecting shaft has a connection portion and a neck that extends from the connecting shaft to a ball. The ball is received within the receptacle of the body and defines a pin passage therethrough. The pin of the insert is received within the pin passage of the ball to prevent translation of the connecting shaft relative to the body.

In aspects, the insert includes retention tabs that extend from side surfaces of the cup. The retention tabs may be received within the walls of the body to retain the insert within the body such that the pin is received within the pin passage.

In another aspect of the present disclosure, a spinal construct includes a spinal rod, screws, and a polyaxial offset lateral connector. The spinal rod defining a longitudinal axis. The screws are configured to secure the spinal rod to a spine of a patient such that the spinal rod extends along the spine. The polyaxial offset lateral connector includes a body, an insert, and a connecting shaft. The body has a base that defines a receptacle and walls that extend from the base to define a saddle. The saddle is configured to secure the body to the spinal rod. The insert includes a cup and a pin that extends from a bottom surface of the cup. The connecting shaft has a connection portion and a neck that extends from the connecting shaft to a ball. The ball is received within the receptacle of the body and defines a pin passage therethrough. The pin of the insert is received within the pin passage of the ball to prevent translation of the connecting shaft relative to the body. The neck extends from the ball in a direction that is substantially transverse to the longitudinal axis.

In aspects, the insert includes retention tabs that extend from side surfaces of the cup. The retention tabs are received within the walls of the body to retain the insert within the body with the pin received within the pin passage.

In another aspect of the present disclosure, a method of securing a spinal construct to a spine of a patient includes securing a first screw to a first vertebra of the spin and securing a second screw to a second vertebra of the spin. With the first and second screws secured, a spinal rod is secured in a head of the first screw and a head of the second screw such that the spinal rod extends along the spine. The spinal rod is also received in a saddle of any of the polyaxial offset lateral connectors detailed above.

In aspects, the method includes securing a third screw to the second vertebra of the spine and receiving a connection portion of a connecting shaft in a head of the third screw.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of a spinal construct including an exemplary embodiment of a polyaxial offset lateral connector provided in accordance with the present disclosure;

FIG. 2 is a perspective view, with parts separated, of the polyaxial offset lateral connector of FIG. 1;

FIG. 3 is a top view of the polyaxial offset lateral connector of FIG. 1; and

FIG. 4 is a cross-sectional view taken along the section line 4-4 of FIG. 3.

### DETAILED DESCRIPTION

Embodiments of the present disclosure are now described in detail with reference to the drawings in which like reference numerals designate identical or corresponding elements in each of the several views. As commonly known, the term “clinician” refers to a doctor, as surgeon, a nurse, or any other care provider and may include support personnel. Additionally, the term “proximal” refers to the portion of the device or component thereof that is closer to the clinician and the term “distal” refers to the portion of the device or component thereof that is farther from the clinician. In addition, the term “cephalad” is known to indicate a direction toward a patient' s head, whereas the term “caudal” indicates a direction toward the patient' s feet. Further still, the term “lateral” is understood to indicate a direction toward a side of the body of the patient, i.e., away from the middle of the body of the patient. The term “posterior” indicates a direction toward the patient's back, and the term “anterior” indicates a direction toward the patient's front. Additionally, terms such as front, rear, upper, lower, top, bottom, and similar directional terms are used simply for convenience of description and are not intended to limit the disclosure. In the following description, well-known functions or constructions are not described in detail to avoid obscuring the present disclosure in unnecessary detail.

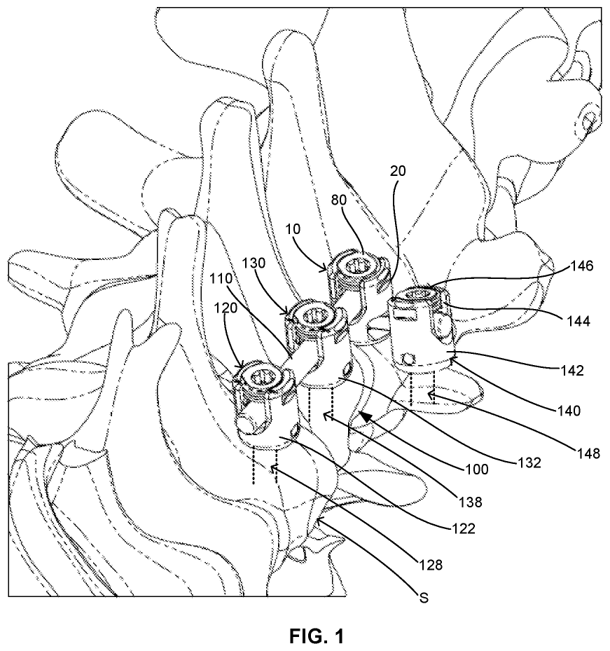

Referring now to FIG. 1, a spinal construct 100 is provided and includes a polyaxial offset lateral connector or connector 10 in accordance with the present disclosure. The spinal construct 100 includes the connector 10, a spinal rod 110, a first screw 120, a second screw 130, and a third screw 140. The spinal rod 110 may be any suitable spinal rod 110 known in the art. The connector 10 allows for adjustment of the spinal construct 100 in a direction transverse to the spinal rod 110.

Each of the first, second, and third screws 120, 130, 140 is a spinal screw having a head, e.g., head 122, 132, 142, and a shank 128, 138, 148. As shown, each head 122, 132, 142 includes a set screw that secures the head to a rod, e.g., spinal rod 110. In some embodiments, one or more of the heads 122, 132, 142 may be a taper lock head that is configured to secure the respective screw 122, 132, 142 to a rod without requiring a set screw. The shank 128, 138, 148 of each of the spinal screws 120, 130, 140 is secured to a portion of a spine S to secure the respective spinal screw 120, 130, 140 to the spine S. For a detail description of an exemplary spinal screw and head, reference can be made to U.S. Pat. No. 8,403,971 and for a detailed description of an exemplary taper lock housing reference can be made to U.S. Pat. No. 8,814,919, the entire contents of each of these patents are hereby incorporated by reference.

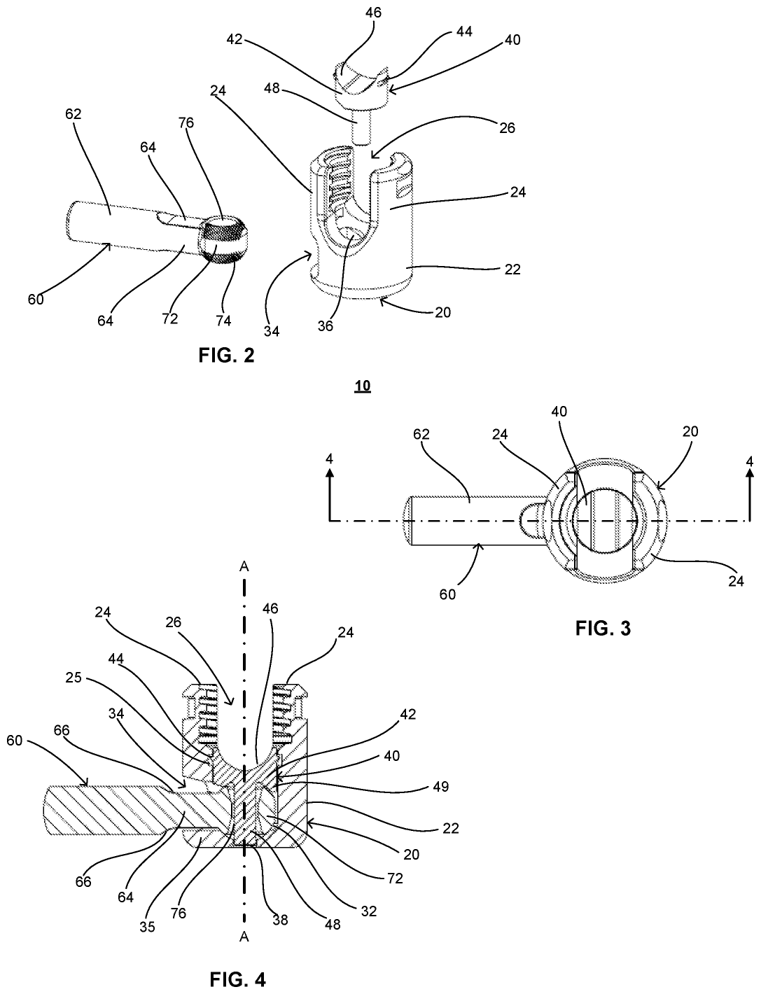

With reference to FIGS. 2-4, the connector 10 includes a body 20, an insert 40, and a connecting shaft 60. The body 20 defines a longitudinal axis A-A and includes a base 22 and a walls 24 extending from the base 22 to define a U-saddle therebetween forming a channel 26. An inner surface of each of the walls 24 defines a recess 25 adjacent the base 22. The inner surface of each of the walls 24 may include a threaded portion configured to threadably receive a set screw 80 (FIG. 1), as detailed below. With particular reference to FIG. 4, the base 22 defines a receptacle 32 that is in communication with the channel 26 via a channel opening 36. The base 22 also defines a pin opening 38 in a bottom portion thereof that is in communication with the receptacle 32. The pin opening 38 may be a blind hole or may pass entirely through the bottom portion of the base 22, as shown. One side of the base 22 defines a side opening 34 that is in communication with the receptacle 32. The side opening 34 is sized and dimensioned to permit a portion of the connecting shaft 60 to pass into the receptacle 32.

The insert 40 includes a cup 42 and a pin 48. The cup 42 is sized and dimensioned to be received within the channel opening 36 defined in the body 20. The cup 42 includes retention tabs 44 extending from side surfaces of the cup 42. The retention tabs 44 are sized and dimensioned to be received within the recesses 25 of the walls 24 of the body 20 to releasably secure the cup 42 within the channel opening 36. The cup 42 also includes a top surface 46 that is curved from a bottom portion of the U-shaped saddle of the body 20. The pin 48 extends from a bottom surface of the cup 42 opposite the top surface 46 and is substantially cylindrical in shape. The insert 40 may also include a locking tab 49 that extends from an edge of the bottom surface of the cup 42 that is positioned opposite of the side opening 34 when the cup 42 is received within the channel opening 36. As detailed below, the locking tab 49 is configured to engage a portion of the connecting shaft 60 to fix the connecting shaft 60 relative to the body 20.

The connecting shaft 60 includes a connecting portion 62, a neck 64 and a ball 72. The connecting portion 62 is generally cylindrical in shape. The neck 64 extends from one end of the connecting portion 62 to the ball 72. The neck 64 has top and bottom flats 66 that are parallel to one another on opposite sides of the neck 64. The top and bottom flats 66 reduce the dimensions of the neck 64 when compared to the connecting portion 62. The ball 72 is sized and dimensioned to be received within the receptacle 32 defined in the base 22 of the body 20. The outer surface of the ball 72 may include engagement features 74 that are configured to engage the walls defining the receptacle 30 to fix the ball 72, and thus the connecting shaft 60, relative to the body 22. Specifically, the engagement features 74 increase friction between the ball 72 and the walls defining the receptacle 50 to resist movement of or fix the connecting shaft 60 relative to the body 22. The ball 72 defines a pin passage 76 that passes through the center of the ball 72 in a direction perpendicular to planes defined by the top and bottom surfaces 66. With particular reference to FIG. 4, the walls defining the pin passage 76 are convex towards the pin passage 76. In some embodiments, the walls defining the pin passage 76 are linear such that the pin passage is cylindrical.

Continuing to refer to FIGS. 2-4, a method of assembling the connector 10 is detailed in accordance with the present disclosure. Initially, the ball 72 of the connecting shaft 60 is inserted through the side opening 34 of the body 20 until the ball 72 is received within the receptacle 30. When the ball 72 is received within the receptacle 30 and is rotatable within the receptacle 30 such that the connecting shaft 60 is movable relative to the base 20. The top and bottom flats 66 of the neck may reduce the diameter of the connecting shaft 60 to allow for additional movement of the ball 72 within the receptacle 30. In addition, the base 22 may include a bottom surface 35 that engages the bottom flat 66 to limit movement of the connecting shaft 60 in at least one direction relative to the body 20. The engagement features 74 may engage the walls defining the receptacle to resist movement of the connection shaft 60 relative to the body 20.

When the ball 72 is received within the receptacle 30, the insert 40 is positioned in the body 20 such that the cup 42 is received in the channel opening 36 and the pin 48 passes through the pin passage 76 defined through the ball 72. When the insert 40 is received in the channel opening 36, the retention tabs 44 are received within the recesses 25 to partially secure the insert 40 within the body 20. As the insert 40 is inserted into the channel opening 36, the retention tabs 44 may engage the walls 24 to flex the walls 24 outward until the retention tabs 44 are received within the recesses 25. When the retention tabs 44 are received within the recess 25, the locking tab 49 may engage an outer surface of the ball 72 when the cup is received within the channel opening 36. The pin 48 extends entirely through the pin passage 76 and is received within the pin opening 38 in the bottom portion of the base 22.

When the insert 40 is received within the channel opening 36, the pin 48 limits movement of the connecting shaft 60 in a direction towards and away from a longitudinal axis of the pin 48, i.e., translation relative to the body 20. The pin 48 also inhibits rotation of the connecting shaft 60 about its longitudinal axis when the body 20 is fixed. In contrast, when the insert 40 is received within the channel opening 36, the connection shaft 60 is pivotable about the pin 48 and is only limited by the interaction of the neck 64 and the walls defining the side opening 34. In addition, as a result of the wall defining the pin passage 76 being convex, the connection shaft 60 is also pivotable in a vertical direction relative to the body 20 limited by the engagement of the top and/or bottom flats 66 of the neck 64 engaging the top and bottom surfaces of the side opening 34 when pivoted transverse to the longitudinal axis of the body 20.. For example, when the walls defining the pin passage 76 are convex, the connecting shaft 60 may be rotatable about its longitudinal axis in a range of about ±5°, may be pivotable up and down (i.e., towards and away bottom surface 35) about a center point of the ball 72 in a range of about ±5°, and may be pivotable about an axis of the pin 48 in a range of about ±35°. In contrast, when the walls of the pin passage 76 are linear, the connecting shaft 60 may be limited to being pivotable about an axis of the pin 48 in a range of about ±35°.

Referring back to FIG. 1, when the connector 10 is assembled, the connector 10 may be secured within a spinal construct, e.g., spinal construct 100. To secure the connector 10 within the spinal construct 100, the connecting portion 62 of the connecting shaft 60 is positioned within a saddle 144 of the third screw 140 and the body 20 is manipulated to position the spinal rod 110 within the channel 26 (FIG. 4) of the body 20. With the connection portion 62 positioned within the saddle 144 of the third screw 140, a set screw 146 is tightened to the head 142 of the third screw 140 to secure the connection portion 62 within the saddle 144 to secure the connection shaft 60 to the third screw 140. With the connecting shaft 60 secured to the third screw 140, the set screw 80 is threaded into the body 20 to secure the spinal rod 110 within the channel 26. As the set screw 80 is tightened in the body 20, the set screw 80 may drive the spinal rod 110 into engagement with the top surface 46 of the insert 40 such that a bottom surface of the cup 42 engages the ball 72 to fix the ball 72, and thus the connecting shaft 60, relative to the body 20. When the spinal rod 110 engages the top surface 46 of the insert 40, the locking tab 49 may engage the outer surface of the ball 72 to fix the ball 72 relative to the body 20.

In some embodiments, the walls 24 of the body 20 include a taper lock feature in place of the threaded set screw arrangement to secure the body 20 to the spinal rod 110.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

1. A polyaxial offset lateral connector comprising:
a body having a base defining a receptacle and walls extending from the base to define a saddle;
an insert including a cup and a pin extending from a bottom surface of the cup; and
a connecting shaft having a connection portion extending away from a ball, the ball received within the receptacle of the body and defining a pin passage therethrough, the pin of the insert received within the pin passage of the ball to prevent translation of the connecting shaft relative to the body.

2. The polyaxial offset lateral connector according to claim 1, wherein the insert includes retention tabs extending from side surfaces of the cup, the retention tabs received within the walls of the body to retain the insert within the body such that the pin received within the pin passage.

3. The polyaxial offset lateral connector of claim 1, wherein the insert includes locking tabs configured to engage a portion of the connecting shaft to fix the connecting shaft relative to the body.

4. The polyaxial offset lateral connector of claim 1, wherein the walls of the body of the polyaxial offset lateral connector includes a threaded inner portion configured to threadably receive a set screw.

5. The polyaxial offset lateral connector of claim 1, wherein the base of the body of the polyaxial offset lateral connector defines a side opening that is in communication with the receptacle, the side opening sized and dimensioned to permit a portion of the connecting shaft to pass into the receptacle.

6. The polyaxial offset lateral connector of claim 1, wherein an outer surface of the ball includes engagement features configured to engage the walls of the body.

7. The polyaxial offset lateral connector of claim 6, wherein the engagement features of the ball are further configured to resist movement of or fix the connecting shaft relative to the body.

8. The polyaxial offset lateral connector of claim 1, further comprising a neck extending from the connecting shaft to the ball.

9. A spinal construct comprising:
a spinal rod defining a longitudinal axis;
one or more screws configured to secure the spinal rod to a spine of patient such that the spinal rod extends along the spine; and
a polyaxial offset lateral connector including:
a body having a base defining a receptacle and walls extending from the base to define a saddle, the saddle configured to secure the body to the spinal rod;
an insert including a cup and a pin extending from a bottom surface of the cup; and
a connecting shaft having a connection portion extending away from a ball, the ball received within the receptacle of the body and defining a pin passage therethrough, the pin of the insert received within the pin passage of the ball to prevent translation of the connecting shaft relative to the body; the connecting portion extending away from the ball in a direction substantially transverse to the longitudinal axis.

10. The spinal construct according to claim 9, wherein the insert includes retention tabs extending from side surfaces of the cup, the retention tabs received within the walls of the body to retain the insert within the body with the pin received within the pin passage.

11. The spinal construct according to claim 9, wherein the insert includes locking tabs configured to engage a portion of the connecting shaft to fix the connecting shaft relative to the body.

12. The spinal construct according to claim 9, wherein the one or more screws includes a taper lock head configured to secure the one or more screw to the spinal rod without a set screw.

13. The spinal construct according to claim 9, wherein the walls of the body of the polyaxial offset lateral connector includes a threaded inner portion configured to threadably receive a set screw.

14. The spinal construct according to claim 9, wherein the base of the body of the polyaxial offset lateral connector defines a side opening that is in communication with the receptacle, the side opening sized and dimensioned to permit a portion of the connecting shaft to pass into the receptacle.

15. The spinal construct according to claim 9, wherein an outer surface of the ball includes engagement features configured to engage the walls of the body.

16. The spinal construct of claim 15, wherein the engagement features of the ball are further configured to resist movement of or fix the connecting shaft relative to the body.

17. The spinal construct of according to claim 9, further comprising a neck extending from the connecting shaft to the ball.

18. A method of securing spinal construct to a spine of a patient, the method comprising:
securing a first screw to a first vertebra of the spine;
securing a second screw to a second vertebra of the spine;
securing a spinal rod in a head of the first screw and a head of the second screw, the spinal rod extending along the spine; and
receiving the spinal rod in a saddle of a polyaxial offset lateral connector, the polyaxial offset lateral connector including:
a body having a base defining a receptacle and walls extending from the base to define the saddle, the saddle configured to secure the body to the spinal rod;
an insert including a cup and a pin extending from a bottom surface of the cup; and
a connecting shaft having a connection portion extending away from a ball, the ball received within the receptacle of the body and defining a pin passage therethrough, the pin of the insert received within the pin passage of the ball to prevent translation of the connecting shaft relative to the body; the connecting portion extending away from the ball in a direction substantially transverse to the longitudinal axis.

19. The method according to claim 18, further comprising securing a third screw to the second vertebra of the spine and receiving the connection portion of the connecting shaft in a head of the third screw.

20. The method according to claim 18, wherein a neck extends from the connecting shaft to the ball.
